(19) United States
(12) Patent Application Publication
(10) Pub. No.: US 2002/0162970 A1
(43) Pub. Date: Nov. 7, 2002

(54) Optical sensing and control of ultraviolet fluid treatment dynamics

(76) Inventor: Michael Sasges, London (CA)

Correspondence Address: GOWLING LAFLEUR HENDERSON LLP, COMMERCE COURT WEST, SUITE 4900, TORONTO, ON, M5L 1J3, CA

(21) Appl. No.: 09/846,682

(22) Filed: May 2, 2001

(51) Int. Cl. G01N 21/01

(52) U.S. Cl. 250/432 R

(57) ABSTRACT

An ultra violet light sterilizing apparatus utilizing a silicon carbide (SiC) photodiode sensor is described The ultraviolet light fluid sterilization apparatus includes a fluid chamber, at leas one ultraviolet light source configured to emit ultraviolet light into the fluid chamber, and at leas one ultraviolet light sensor that includes a silicon carbide photodiode. Each UV light sensor includes a sealed outer housing having an optically transparent window. A silicon carbide photodiode is located inside the housing adjacent the transparent window. Each UV light sensor also includes a signal amplification unit that includes an amplifier mounted on a printed circuit board located inside the housing. The UV sterilization apparatus also includes a controller configured to receive, as input, a signal from each ultraviolet light sensor. The controller compares the input signal to a desired UV light intensity and outputs a control signal to each ultraviolet light source to adjust the, intensity of the ultraviolet light emitted from each ultraviolet light source.

### BACKGROUND OF THE INVENTION

This invention relates generally to ultraviolet light treatment of fluids and more particularly, to optical sensing and control of ultraviolet light intensity levels in an ultraviolet light fluid treatment process with silicon carbide photodiode detectors.

Ultraviolet (UV) light may be used to sterilize water and other fluids. The ultraviolet light disrupts the DNA of microorganisms in the fluid, which prevents reproduction and thus kills the microorganisms. Regulation of UV light intensity is important in the UV light sterilization process because a minimum level of ultraviolet light intensity is typically required. Also, excessive levels of UV light intensity can result in high maintenance and higher operation cost.

Photodiodes are sometimes used to detect and regulate UV light intensity in such sterilization processes. The UV light spectrum includes wavelengths from 100 to 400 nanometers (nm). However, typical photodiodes used to measure UV light in the known UV light sterilization processes are usually broad range wavelength detectors. For example, UV enhanced silicon detectors are used which are sensitive to light wavelengths ranging from 200 to 1100 nm . Gallium arsenide phosphide and gallium phosphide detectors, which are sensitive to light wavelengths ranging from 200 to 650 nm, are also use. These devices are inherently sensitive to visible light waves in addition to the UV spacing When only UV light detection is desired and outside light sources such as visible light are also present, erroneous signals may result.

Therefore, filters are needed to block Out wavelengths longer than 400 nm to eliminate erroneous signals triggered by other light sources. However, filters are prone to degradation which can lead to permitting light outside the UV spectrum into the detector, which results in false and inaccurate readings, In short, filters are costly, complex and are prone to being unstable. Additionally, filter degradation results in costly maintenance and/or equipment downtime.

It would be desirable to provide an ultraviolet light sterilization process incorporating a UV detector that is not sensitive to light outside the V spectrum to eliminate erroneous signals caused by non-UV light sources. It would her be desirable to provide an ultraviolet light sterilization process that does not depend upon light filters thereby lowering maintenance costs and equipment downtime due to filter repair or replacement

### SUMMARY OF THE INVENTION

These and other objects may be attained by an ultra violet light sterilizing apparatus utilizing a silicon carbide (SiC) photodiode sensor. The ultraviolet light fluid sterilization apparatus includes a fluid chamber, at least one ultraviolet light source configured to emit ultraviolet light into the fluid chamber, and at least one ultraviolet light sensor that includes a silicon carbide photodiode.

Each UV light sensor includes a sealed outer housing having an optically transparent window. A silicon carbide photodiode is located inside the housing adjacent the transparent window. The housing also includes at least one sealable outlet to permit electrical wire connections to pass into the housing. The optically transparent window may be fabricated from sapphire or fused silica, for example.

The UV light fluid station apparatus further includes a controller for sampling the sign from each ultraviolet light sensor. The controller compares the sampled signals to a desired UV light intensity and outputs a control signal to each ultraviolet light source to adjust the intensity of the ultraviolet light emitted from each ultraviolet light source. The controller could also be used to monitor for a minimum intensity and trigger an alarm.

In operation, fluid flows into the chamber (or treatment zone) of the ultraviolet light sterilization apparats. The fluid is then irradiated with UV light from the ultraviolet light source. The UV light sensor measures the intensity of the UV light inside the chamber of the apparatus and the controller samples the signal generated by the sensor. Particularly, the SiC photodiode senses the intensity of the UV light inside the chamber, a signal amplification unit amplifies the signal, and the controller samples the signal generated by the sensor. The controller ascertains any difference between the sensed V intensity and a predetermined desired UV intensity. The controller then adjusts the intensity of the ultraviolet light source to correspond to the predetermined level of UV intensity.

The above described ultraviolet light fluid sterilization apparatus utilizes a silicon carbide UV detector that is not sensitive to light outside the UV spectrum and thus eliminates erroneous signal caused by non-UV light sources. Particularly, the silicon carbide UV sensor has good UV sensitivity in the 200 to 300 nm band and is insensitive to infrared radiation above 400 nm Also because the silicon carbide sensor is not sensitive to light having wavelengths greater than 400 nm, light filters are not required to filter non-UV light.

### BRIEF DESCRIPTION OF THE DRAWINGS

Embodiments of the present invention will be described with reference to the accompanying drawings, wherein like numerals designate like elements, and in which:

FIG. 1 is a schematic view of an ultraviolet light fluid sterilization apparatus in accordance with one embodiment of the present invention;

FIG. 2 is a sectional side view of the silicon carbide ultraviolet light sensor shown in FIG. 1;

FIG. 2a and FIG. 2b are schematic illustrations of a means by which the present sensor may be mounted im a fluid treatment chamber or system;

FIG. 3 is a side elevation of a radiation source module comprising the present senor;

FIG. 4 is a schematic illustration of the present sensor mounted in a submersed construction with respect to the fluid treatment system; and

FIG. 5 is a schematic illustration of the present sensor mounted in a non-submersed conduction with respect to the fluid treatment system.

### DETAILED DESCRIPTION OF THE PREFERRED EMBODIMENTS

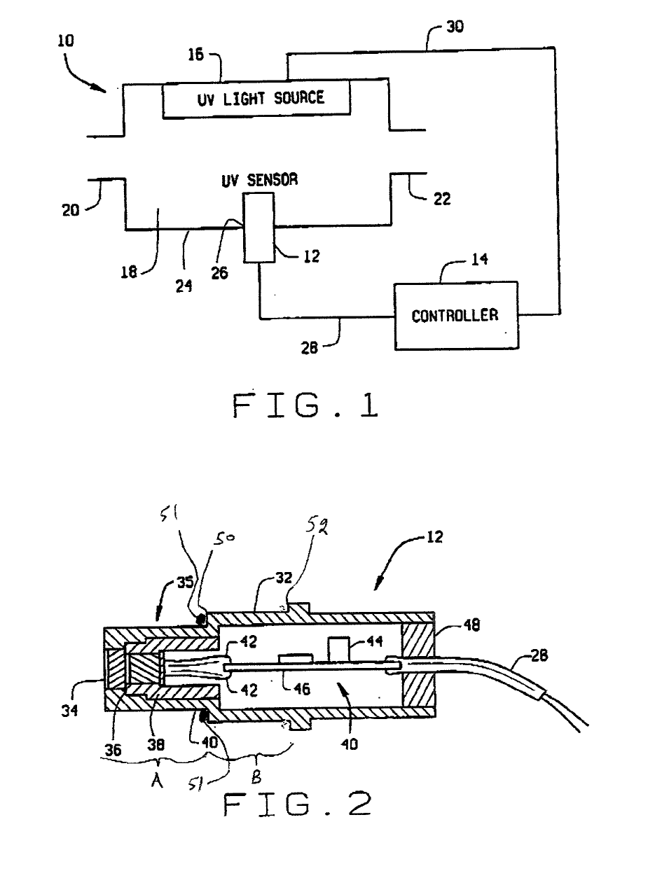

FIG. 1 is a schematic view of an ultraviolet light fluid sterilization apparatus 10 in accordance with an embodiment of the present invention. Apparatus 10 sterilizes or disinfects various fluids, for example, water, by delivering concentrated doses of ultraviolet energy to the fluid The ultraviolet energy, also called UV light, disrupts the DNA of microorganisms in the fluid preventing reproduction and thus killing the microorganisms To successfully sterilize a fluid a minimum level of ultraviolet light exposure is required. Apparatus 10 includes an ultraviolet light sensor 12 to measure UV light intensity and a controller 14 to ascot any difference between the sensed UV intensity and a predetermined desired intensity and to adjust the output of an ultraviolet light source 16 to achieve the desired level of UV light intensity.

Apparatus 10 also includes a chamber 18 having an inlet 20 and an outlet 22. Chamber 18 is a tube trough which the fluid may continuously flow. Alternatively, chamber 18 may be a tank which permits a longer dwell time of the fluid in chamber 18. A tank permits batch processing of the fluid, while a tube generally is used for continuous processing of the fluid as it flows through chamber 18.

UV light source 16 is located inside chamber 18. However, UV light source 16 may be located outside chamber 18 adjacent an opening or window in chamber 18 that permits the UV light emitted from light source 16 to enter chamber 18.

Silicon carbide UV light sensor 12 is mounted on a wall 24 of chamber 18. Particularly, sensor 12 extends through an opening 26 in wall 24 so that at least a portion of sensor 12 is located inside chamber 18. However in an alternative embodiment, UV light sensor 12 may be mounted outside chamber 18 adjacent an opening or window permitting UV light to pass from inside chamber 18 to sensor 12.

Silicon carbide UV light sensor 12 and UV light source 16 are each electrically connected to controller 14. Particularly, sensor 12 is coupled to controller 14 by a controller input line 28. UV light source 16 is coupled to controller 14 by a controller output line 30.

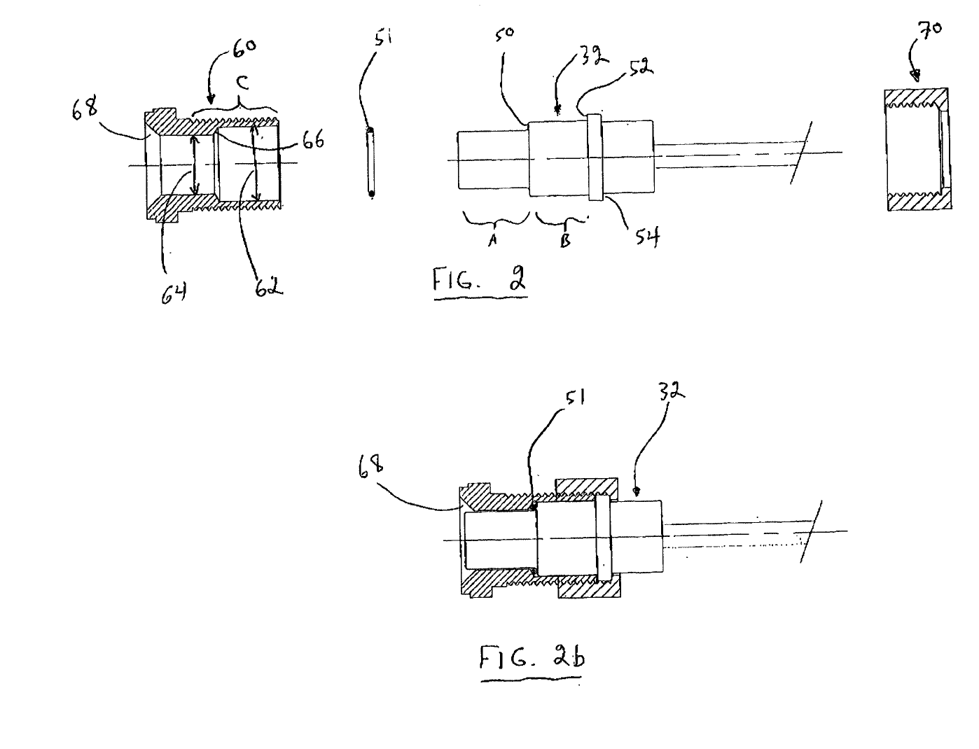

FIG. 2 is a sectional side view of silicon carbide ultraviolet light sensor 12. UV sensor 12 includes a sealed housing 32. An optically transparent window 34 is located in a first end 35 of housing 32. Optically transparent window 34 permits UV light to enter housing 32. Window 34 may be fabricated from any suitable material, for example sapphire or fused silica A silicon carbide photodiode 36 is located inside housing 34 adjacent to window 34. SiC photodiode 36 is sensitive to light in the ultraviolet spectrum of about 200 to about 400 nm. Photodiode 36 is not sensitive to infrared and visible light having wavelengths greater than 400 nm Because SiC photodiode 36 is not sensitive to light outside the UV spectrum, stray non-UV light entering chamber 18 (shown in FIG. 1) will not cause erroneous readings by sensor 12 of the UV light intensity inside chamber 18. A TEFLON® (polytetrafluoroethylene) plastic insert 38, configured to fit inside fist end 36 of housing 32 and engage wall 40 of housing 32, holds photodiode 36 in place adjacent window 34.

Sensor 12 also includes a signal amplification unit 40 electrically connected to photodiode 36 by conductors 42. Signal amplification unit 40 includes a signal amplifier 44 mounted on a printed circuit board 46. Circuit board 46 is mounted inside housing 32. Conductors 42 are electrically connected to signal amplifier 44 through printed circuit board 46. Controller input line 28 enters housing 32 through a sealable inlet 48. Controller input line 28 is electrically connected to signal amplifier 44 through printed circuit board 46. In an alternate embodiment, signal amplification unit 40 is located outside and separate from housing 32.

As illustrated, sealed housing 32 comprises a first step 50, a second step 52 and a third step 54. With reference to FIG. 2 sealed housing 32 preferably has a diameter of about 0.500 inches in region A and a diameter of 0.649 inches in region B.

With reference to FIG. 2a, there is illustrated a coupling portion 60 comprising a threaded portion C. As illustrated, coupling portion 60 comprises a first internal diameter 62 and a second internal diameter 64. The internal sections of coupling portion 60 corresponding to first internal diameter 62 and second internal diameter 64 are separated by a shoulder 66. Preferably, first inter diameter 62 is 0.655 inches and second internal diameter 64 is 0.520. As shown, coupling portion 60 comprised a flanged end 68 for receiving a UV radiation transparent window (not shown).

Sensor 12 may be mounted in threaded aperture in the wall (not shown) of a UV chamber or treatment system in the following maser

With reference to FIGS. 2a and 2b, coupling portion 60 is welded to (or otherwise attached in a substantially fluid-tight manner) the wall of a UV chamber or treatment system such that flanged end 68 of coupling portion 60 faces the interior of the UV chamber or treatment system initially, an O-ring 51 or other sealing member is placed on the exterior of sealing housing 32 such that O-ring 51 rests against first step 50 of sealed housing 32. From the outside of the UV chamber, sealed housing 32 is inserted into coupling portion 60 until second step 52 abuts the end face of coupling portion 60. This contact limits the compression of O-ring 51. Finally, a nut 70 is placed over the rear of sealed housing 32, until the nut presses against third step 54 of sealed housing 32. Nut 70 engages with the threaded portion C of coupling portion 60, ensuring that sealed housing 32 remains fully inserted into coupling portion 60, compressing O-ring 51 and maintaining a substantially fluid-tight seal.

In operation, fluid flows into chamber 18 of ultraviolet light sterilization apparatus 10. The fluid is then irradiated with UV light from ultraviolet light source 16. UV light sensor 12 is responsive to the intensity of the UV light inside chamber 18. Particularly, SiC photodiode 36 generates a signal representative of the intensity of the UV light inside chamber 18. The generated signal is amplified by signal amplifier 44 of signal amplification unit 40, and controller 14 samples the amplified signal on input line 28. Controller 14 ascertains any difference between the sensed UV intensity and a predetermined desired UV intensity. Controller 14 then sends a control signal through controller output line 30 to adjust the intensity of ultraviolet light source 16 to correspond to the predetermined UV intensity level. For examples if the sensed UV intensity inside chamber 18 is below the desired UV intensity controller 14 causes UV light source 16 to increase UV light output or to send an

The above described ultraviolet light fluid sterilization apparatus 10 utilizes silicon carbide UV sensor 12 that is not sensitive to light outside the UV spectrum. Because sensor 12 is not sensitive to light outside the UV spectrum, there are no erroneous signals from sensor 12 caused by non-UV radiation. Erroneous signals from sensor 12 could cause controller 14 to adjust the UV intensity too low for proper sterilization. By elimination of erroneous UV intensity signals, SiC photodiode sensor 12 ensures that UV light sterilization apparatus 10 operates efficiently to produce sufficiently sterilized fluids such as water. Additionally, because SiC photodiode sensor 12 is not sensitive to light outside the UV spectrum, the use of light filters to eliminate non-TV light is not required

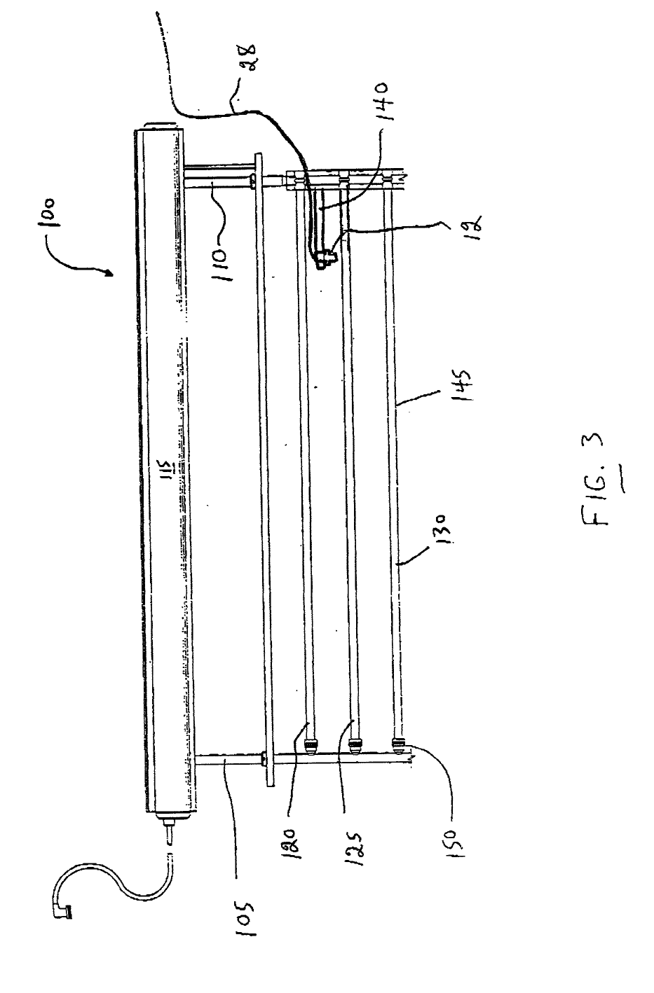

With reference to FIG. 3, there is illustrated a radiation source module 100 incorporating an embodiment of the present sensor. Radiation source module 100 is typically used in an open channel having water in need of treatment (e.g., disinfection) flowing therethrough by gravity. Radiation source module 100 comprises a pair of support legs 105,110 depending from a crosspiece 115. Disposed between support legs 105,110 are a trio of radiation source assemblies 120,125,130. Each radiation source assembly 120,125,130 comprises a radiation source (not shown for clarity)—e.g., all Ultraviolet emitting lamp—disposed within a protective sleeve 145 (e.g., typically made of quartz). Preferably, each protective sleeve 145 is connected to support leg 105 via a coupling nut 150. The design of support legs 105,110 and radiation source assemblies 120 is preferably as is described in one or more of the following:

U.S. Pat. No. 4,872,980;

U.S. Pat. No. 5,006,244;

U.S. Pat. No. 5,019,256;

U.S. patent application Ser. No. 09/185,813;

U.S. patent application Ser. No. 09/258,142;

International patent application S.N. PCT/CA00/00192;

International patent application S.N. PCT/CA00/01001;

International patent application S.N. PCT/CA00/01002;

International patent application S.N. PCT/CA00/01475;

International patent application S.N. CA00/01119; and

U.S. patent application Ser. No. 60/211,971.

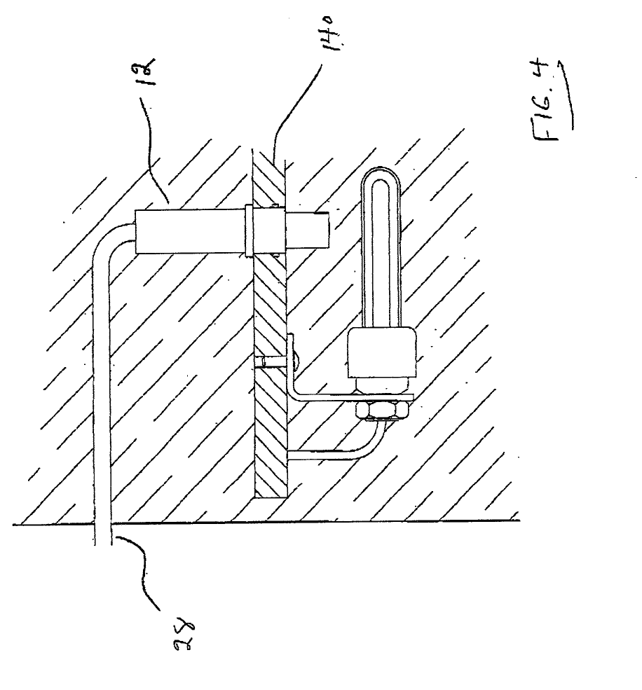

UV light sensor 12 is mounted to support leg 110 via a bracket 140. As will be apparent to those of skill in the art, in this embodiment UV light sensor 12 is constructed to be water impermeable so that it may be fully submersed in the fluid being treated. Submersion of UV light sensor 12 is illustrated schematically in FIG. 4.

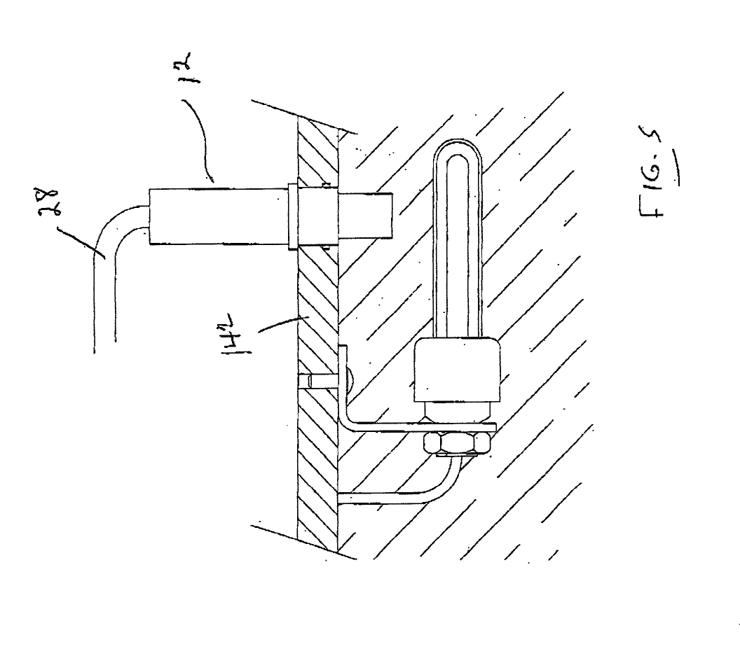

Alternatively, UV light sensor 12 may be used in a so-called “closed” fluid treatment system wherein fluid in seed of treatment flows through a closed conduit or the like. In his case, UV light sensor 12 may be mounted in a wall 142 of the closed conduit or the like—see FIG. 5. Non-limited examples of such “closed” fluid treatment systems in which the present senor maybe advantageously used may be found in U.S. Pat. No. 5,504,335 and International patent application S.N. PCT/CA00/01120.

From the preceding description of various embodiments of the present invention, it is evident that the objects of the invention are attained. Although the invention has been described and illustrated in detail, it is to be clearly understood that the same is intended by way of illustration and example only and is not to be taken by way of limitation. Accordingly, the spirit and scope of the invention are to be limited only by the terms of the appended claims.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. An ultraviolet light fluid sterilizing apparatus comprising:
at least one ultraviolet light source configured to irradiate a fluid with ultraviolet light to sterilize the fluid;
an ultraviolet light sensitive silicon carbide photodiode, said photodiode capable of generating a signal proportional to the intensity of ultraviolet light detected by said photodiode; and
a sealed outer housing comprising an optically transparent window, said silicon carbide photodiode located inside said housing and adjacent said transparent window

2. An apparatus in accordance with claim 1 flirter comprising a signal amplification unit coupled to said silicon carbide photodiode.

3. An apparatus in accordance with claim 2 wherein said signal amplification unit comprises an amplifier mounted on a printed circuit board, said printed circuit board located inside said housing.

4. An apparatus in accordance with claim 1 wherein said silicon carbide photodiode is sensitive to light having a wavelength ranging from about 200 to about 400 nanometers.

5. An apparatus in accordance with claim I wherein said optically transparent window comprises sapphire or quartz.

6. An apparats in accordance with claim 1 wherein said housing farther comprises at least one sealable outlet to permit electrical wire connections to pass through said housing.

7. An ultraviolet light fluid sterilization apparatus comprising:
a fluid chamber,
at least one ultraviolet light source configured to emit ultraviolet light into said fluid chamber, and
at least one ultraviolet light sensor comprising a silicon carbide photodiode.

8. An apparatus in accordance with clam 7 wherein said ultraviolet light sensor further comprises:
a sealed outer housing comprising an optically transparent window, said silicon carbide photodiode located inside said housing and adjacent said transparent window; and
a signal amplification unit coupled to said silicon carbide photodiode.

9. An apparatus in accordance with claim 8 wherein said signal amplification unit comprises an amplifier.

10. An apparatus in accordance with claim 9 wherein said amplifier is mounted on a printed circuit board, said printed circuit board located inside said housing.

11. An apparatus in accordance with claim 7 wherein said silicon carbide photodiode is sensitive to light having a wavelength ranging from about 200 to about 400 nanometers.

12. An apparatus in accordance with claim 7 wherein said optically transparent window comprises sapphire or quartz.

13. An apparatus in accordance with claim 7 wherein said housing firer comprises at least one sealable outlet to permit electrical wire connections to pass though said housing.

14. An apparatus in accordance with claim 13 further comprising a controller configured to receive, as input, a signal from said ultraviolet light sensor and to output a control signal to said ultraviolet light source to control the intensity of the ultraviolet light emitted from said ultraviolet light source.

15. A method of sterilizing a fluid utilizing an ultraviolet light fluid sterilization apparatus, the sterilization apparatus comprising a fluid chamber, at least one ultraviolet light source, and at least one ultraviolet light sensor, each ultraviolet light source configured to emit ultraviolet light into the fluid chamber, and each ultraviolet light sensor comprising a silicon carbide photodiode, said method comprising the steps of:
flowing a fluid into the chamber of the ultraviolet light sterilization apparatus;
irradiating the fluid with ultraviolet light from the at least one ultraviolet light source of the sterilization apparatus;
measuring the intensity of the ultraviolet light in the fluid chamber with the ultraviolet light sensor;
sensing an output signal from the ultraviolet light sensor with the controller; and
adjusting the level of ultraviolet light intensity in the chamber with an output signal from the controller to the light source.

16. A method in accordance with claim 15 wherein each ultraviolet light sensor fleer comprises:
a sealed outer housing comprising an optically transparent window, the silicon carbide photodiode located inside the housing and adjacent the transparent window and
a signal amplification nit.

17. A method in accordance with claim 16 wherein signal amplification unit comprises an amplifier mounted on a printed circuit board, the printed circuit board located inside the housing.

18. A method in accordance with claim 16 wherein the silicon carbide photodiode is sensitive to light having a wavelength ranging from about 200 to about 400 nanometers.

19. A method in accordance with claim 16 wherein the optically transparent window rises sapphire or quartz.

20. A method in accordance with claim 16 wherein said housing further comprises at least one sealable outlet to permit electrical wire connections to pass through said housing.

21. An apparatus in accordance with claim 1 firer comprising a fluid chamber having an interior, said sealed outer housing coupled to said fluid chamber with said transparent dow in optical cooperation with said interior of said fluid chamber, and said at least one violet light source configured to emit ultraviolet light into said fluid chamber.
